(19) United States
(12) Patent Application Publication
(10) Pub. No.: US 2007/0276190 A1
(43) Pub. Date: Nov. 29, 2007

(54) Surgical Retractor, And Method For Establishing An Enlarged Operating Portal In A Patient

(76) Inventor: Paul Tsahakis, Charlotte, NC (US)

Correspondence Address: SCHWARTZ LAW FIRM, P.C., 6100 FAIRVIEW ROAD, SUITE 11350, CHARLOTTE, NC, 28210, US

(21) Appl. No.: 10/582,263

(22) PCT Filed: Dec. 10, 2004
(86) PCT No.: PCT/US04/41792

§ 371 Date: Jan. 3, 2007

Related U.S. Application Data
(60) Provisional application No. 60/528,434, filed on Dec. 10, 2003.

(51) Int. Cl. A61B 1/32

(52) U.S. Cl. 600/210

(57) ABSTRACT

A surgical retractor includes an open-ended hollow sleeve adapted for inserting into a body of a patient at an operating site to hold back surrounding muscle and body tissue. The sleeve defines an enlarged operating portal sufficient to allow entry and manipulation of surgical instruments inside the body. The sleeve has a width dimension of at least 50 mm. The width dimension accommodates multiple side by side dilators adapted for stretching the muscle and body tissue at the operating site. The surgical retractor is inserted into the body over the multiple dilators, and the dilators being subsequently removed to establish the enlarged operating portal.

### TECHNICAL FIELD AND BACKGROUND OF THE INVENTION

This invention relates broadly to surgical retractors, and more specifically to an open-ended, rigid, generally oval-shaped sleeve especially applicable for holding back muscle and body tissue during bilateral spine surgery. The invention creates an enlarged operating portal applicable for receiving a wide variety of surgical instruments. The enlarged portal allows direct visualization of the bone and substantial unobstructed manipulation of the operating instruments at the surgery site. The invention is quickly installed and is minimally invasive.

### SUMMARY OF INVENTION

Therefore, it is an object of the invention to provide an improved surgical retractor especially applicable for bilateral spine surgery.

It is another object of the invention to provide a surgical retractor which is applicable for quickly and conveniently creating an enlarged and stable operating portal to the bone.

It is another object of the invention to provide a surgical retractor which comprises an open-ended, rigid, generally oval-shaped sleeve which has a width dimension of approximately 50 mm and a depth dimension of approximately 25 mm.

It is another object of the invention to provide a surgical retractor which is installed using a pair of guide wires and adjacent serial dilators.

It is another object of the invention to provide a surgical retractor which can be installed at the surgery site in less than three minutes.

It is another object of the invention to provide a surgical retractor which allows substantial manipulation of operating instruments at the surgery site.

It is another object of the invention to provide a surgical retractor which enables direct visualization of the bone.

It is another object of the invention to provide a surgical retractor which allows broad access to the surgery site with little damage to the muscle surrounding the spine.

It is another object of the invention to provide a surgical retractor which permits insertion of angle-tipped instruments directly into the surgery site.

It is another object of the invention to provide a surgical retractor which offers increased visual acuity and flexibility during surgery.

It is another object of the invention to provide a method of forming an enlarged surgical port in the body of a patient.

These and other objects of the present invention are achieved in the preferred embodiments disclosed below by providing a surgical retractor including an open-ended hollow sleeve adapted for inserting into a body of a patient at an operating site to hold back surrounding muscle and body tissue. The sleeve defines an enlarged operating portal sufficient to allow entry and manipulation of surgical instruments inside the body. The sleeve has a width dimension of at least 50 mm. The width dimension accommodates multiple side by side dilators adapted for stretching the muscle and body tissue at the operating site. The surgical retractor is inserted into the body over the multiple dilators, and the dilators being subsequently removed to establish the enlarged operating portal.

According to another preferred embodiment of the invention, the sleeve has a generally oval cross-section.

According to another preferred embodiment of the invention, the sleeve has a generally hourglass-shaped cross-section.

According to another preferred embodiment of the invention, the width dimension of the sleeve is at least two times a depth dimension of the sleeve.

According to another preferred embodiment of the invention, the sleeve has a maximum depth dimension of at least 25 mm. The term “maximum depth dimension” refers herein to a point of greatest depth in the hollow sleeve.

According to another preferred embodiment of the invention, the sleeve has a length of between 40 and 80 mm.

According to another preferred embodiment of the invention, a handle is formed with the sleeve for manipulating the surgical retractor inside the body of the patient.

According to another preferred embodiment of the invention, the sleeve is constructed of a rigid shape-retaining material.

According to another preferred embodiment of the invention, a reinforcing perimeter flange is located at one open end of the sleeve.

In another embodiment, the invention is a method of forming an enlarged operating portal in a body of a patient. The method includes the steps of inserting a first dilator into the body at an insertion site. A second dilator is inserted into the body at an adjacent insertion site. An open-ended hollow sleeve is then brought down over the first and second dilators, and into the body at the insertion sites. The first and second dilators are then removed from the body, such that the hollow sleeve defines an enlarged and stable operating portal sufficient to allow entry and manipulation of surgical instruments inside the body at an operating site.

According to another preferred embodiment of the invention, the method includes inserting a guide wire through the skin for guiding insertion of the first dilator into the body.

According to another preferred embodiment of the invention, the method includes inserting a guide wire through the skin for guiding insertion of the second dilator into the body.

In yet another embodiment, the method includes inserting a first assembly of progressively larger serial dilators into the body at an insertion site, and inserting a second assembly of progressively larger serial dilators into the body at an adjacent insertion site. The open-ended hollow sleeve is then brought down over outermost ones of the first and second assemblies of serial dilators, and into the body at the insertion sites. The first and second assemblies of serial dilators are removed from the body, such that the hollow sleeve defines an enlarged and stable operating portal sufficient to allow entry and manipulation of surgical instruments inside the body at an operating site.

### BRIEF DESCRIPTION OF THE DRAWINGS

Some of the objects of the invention have been set forth above. Other objects and advantages of the invention will appear as the description proceeds when taken in conjunction with the following drawings, in which:

FIGS. 1-4 are views of an improved surgical retractor according to one preferred embodiment of the present invention;

FIGS. 5-8 are views of the surgical retractor according to a second preferred embodiment;

FIGS. 9 and 10 are respective side and end views showing a portion of the patient's spine;

FIGS. 11 and 12 are respective side and end views showing a portion of the patient's spine, and demonstrating insertion of the guide wires through the skin at respective insertion sites;

FIGS. 13 and 14 are respective side and end views showing a portion of the patient's spine, and demonstrating insertion of the first-diameter (innermost) dilators over the guide wires and to the bone;

FIGS. 15 and 16 are respective side and end views showing a portion of the patient's spine, and demonstrating insertion of the second-diameter dilators over the guide wires and to the bone;

FIGS. 17 and 18 are respective side and end views showing a portion of the patient's spine, and demonstrating insertion of the third-diameter dilators over the guide wires and to the bone;

FIGS. 19 and 20 are respective side and end views showing a portion of the patient's spine, and demonstrating insertion of the fourth-diameter dilators over the guide wires and to the bone;

FIGS. 21 and 22 are respective side and end views showing a portion of the patient's spine, and demonstrating insertion of the fifth-diameter dilators over the guide wires and to the bone;

FIGS. 23 and 24 are respective side and end views showing a portion of the patient's spine, and demonstrating insertion of the sixth-diameter dilators over the guide wires and to the bone;

FIGS. 25 and 26 are respective side and end views showing a portion of the patient's spine, and demonstrating insertion of the seventh-diameter dilators over the guide wires and to the bone;

FIGS. 27 and 28 are respective side and end views showing a portion of the patient's spine, and demonstrating insertion of the eight-diameter (outermost) dilators over the guide wires and to the bone;

FIG. 29 is a top view of a portion of the spine, and showing the adjacent openings formed by the multiple serial dilators (shown in phantom);

FIGS. 30-33 are sequential views illustrating the process of bringing the surgical retractor down over the side by side assemblies of serial dilators, and then subsequently removing the assemblies to form the operating portal;

FIG. 34 is a top view showing two surgical retractors located on opposite sides of the spine with respective assemblies of dilators shown in phantom;

FIG. 35 is a top view showing two surgical retractors located on opposite sides of the spine with respective assemblies of dilators removed;

FIG. 36 is a top view corresponding to FIG. 35, but incorporating a further embodiment of the surgical retractor;

FIGS. 37-43 illustrate a third embodiment of the present surgical retractor; and

FIGS. 44-47 illustrate a fourth embodiment of the present surgical retractor.

### DESCRIPTION OF THE PREFERRED EMBODIMENT AND BEST MODE

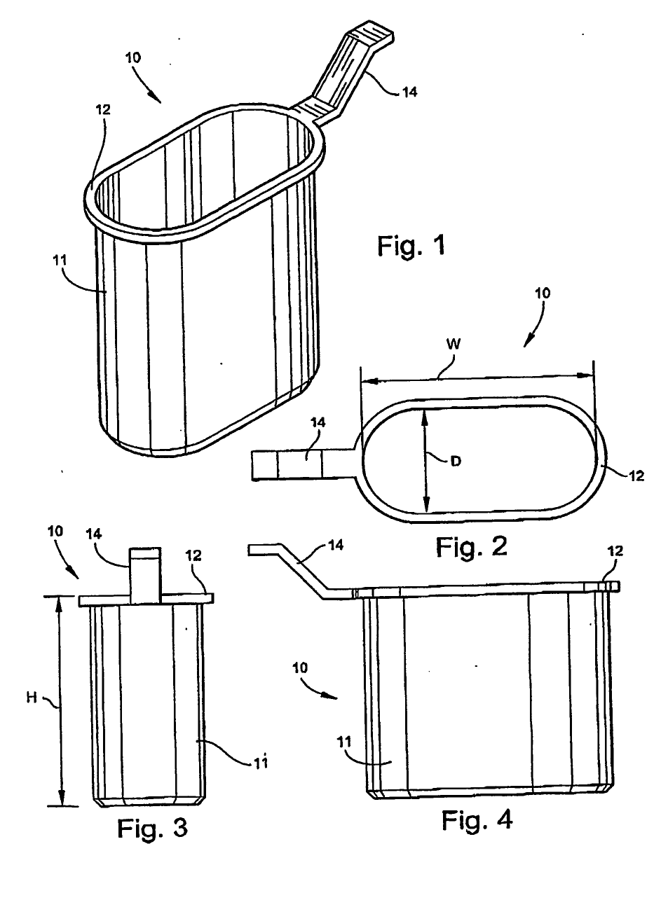

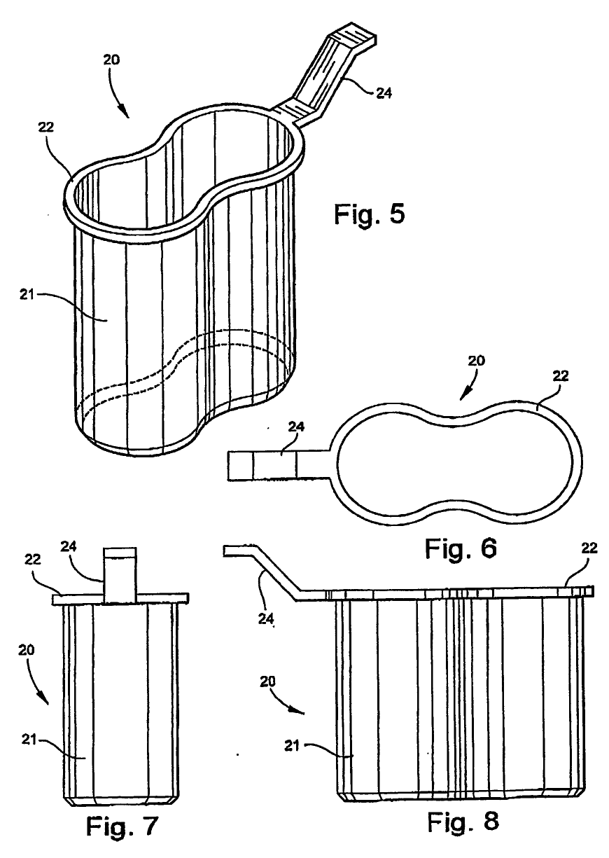

Referring now specifically to the drawings, a surgical retractor according to one preferred embodiment of the invention is illustrated in FIGS. 1-4, and shown generally at reference numeral 10. The retractor 10 comprises an open-ended, rigid, generally oval-shaped hollow sleeve 11 which may be molded or formed of any shape-retaining material capable of sterilization and suitable for insertion into the body. A second embodiment of the present retractor 20 with generally contoured side walls is shown in FIGS. 5-8. The retractor 20 comprises a hollow rigid sleeve 21 with a generally hourglass-shaped cross-section, and is slightly less invasive than the retractor 10 comprising a generally oval-shaped cross-section. As indicated above; the retractor 10, 20 is especially applicable for use in bilateral spine surgery. Preferably, in each embodiment of the retractor 10, 20, a reinforced perimeter flange 12, 22 provides added support strength to protect against collapsing once the retractor 10, 20 is inserted into the body. The retractor 10, 20 may also include a convenient handle 14, 24 in order to facilitate its manipulation by the surgeon at the operating site.

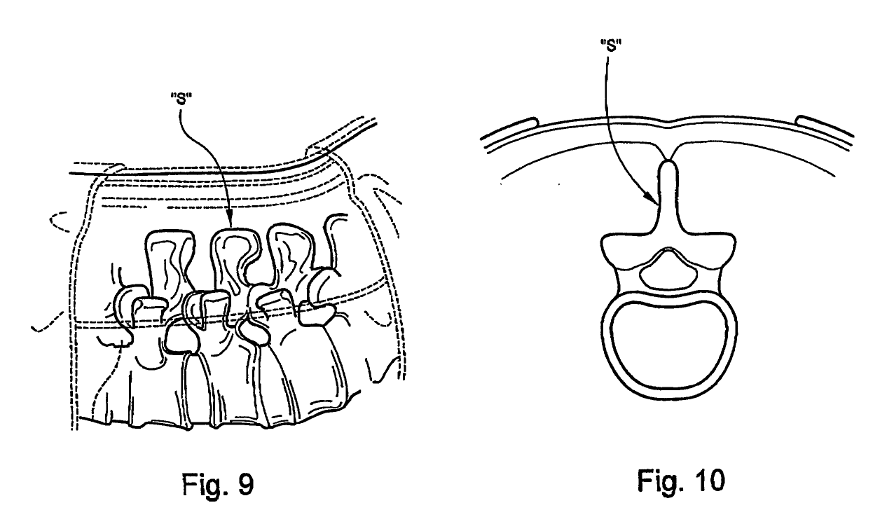

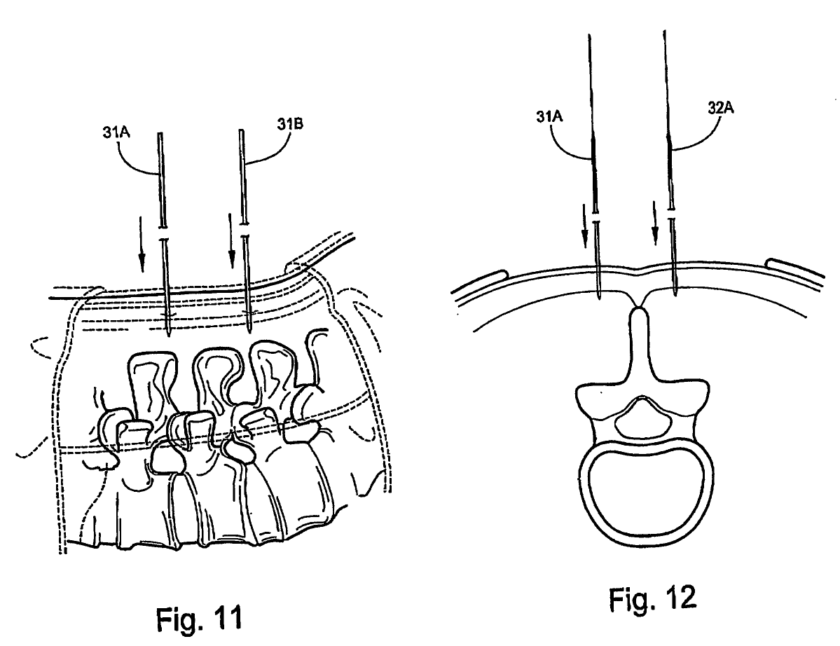

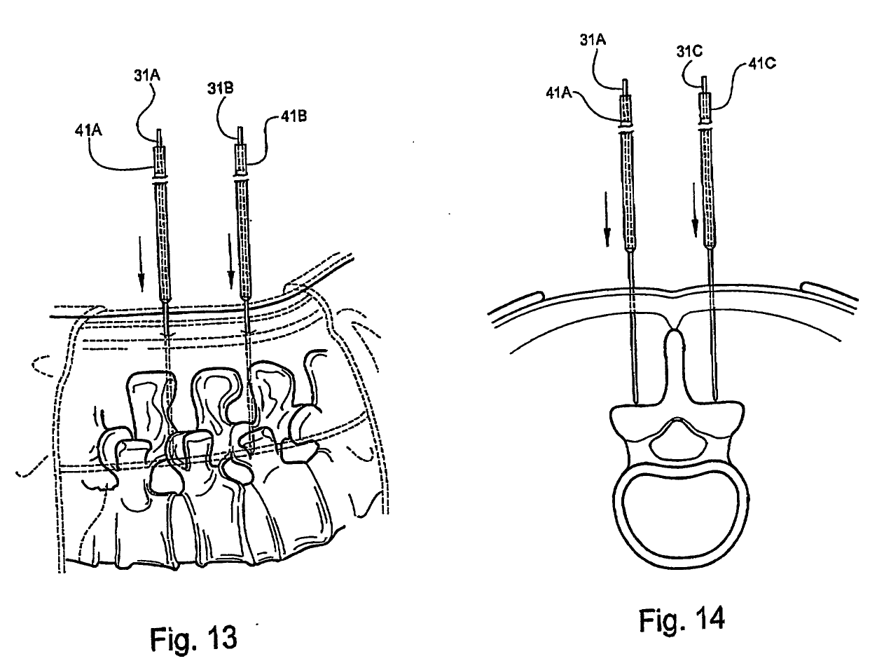

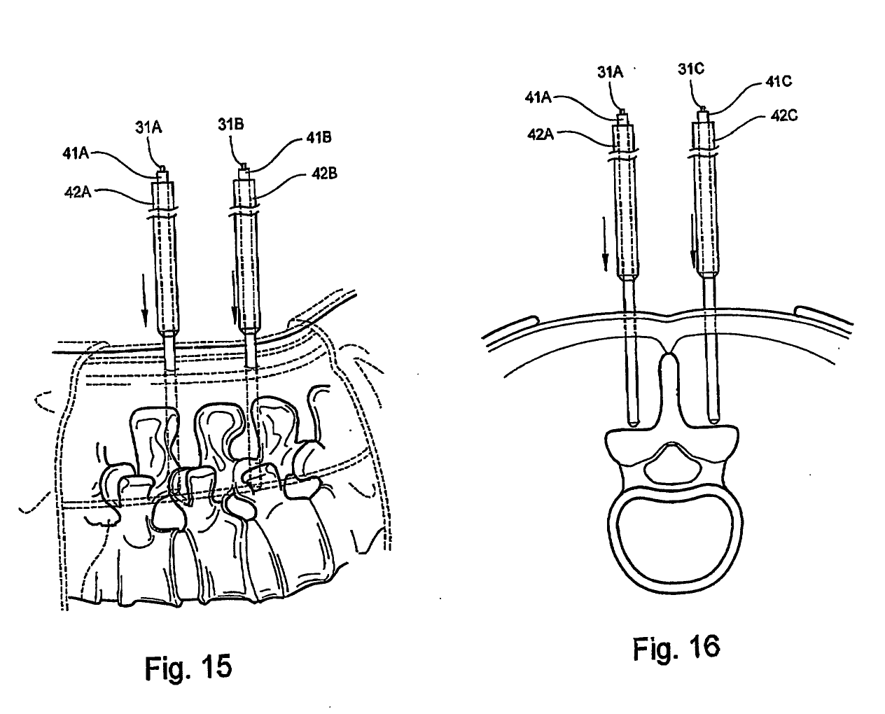

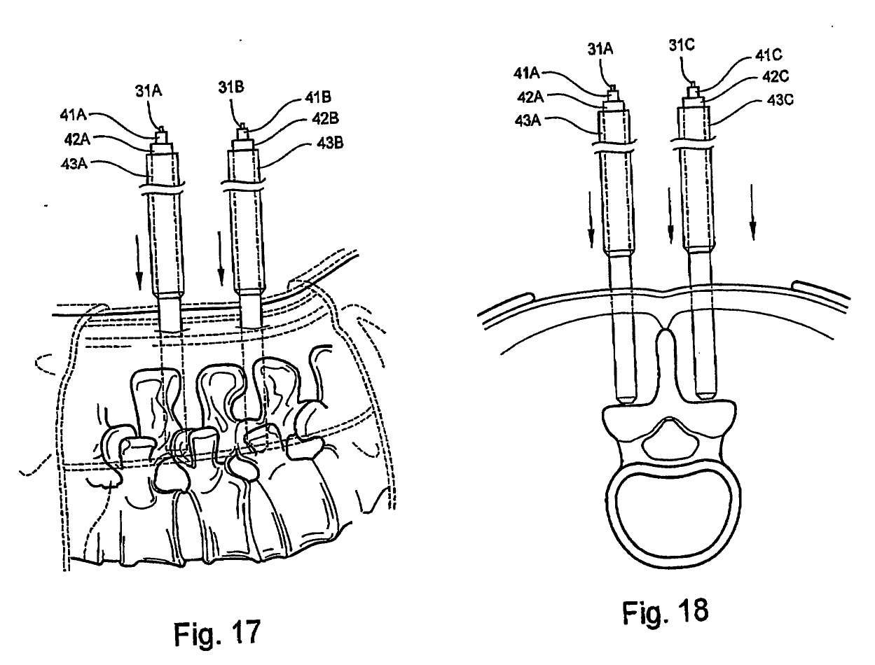

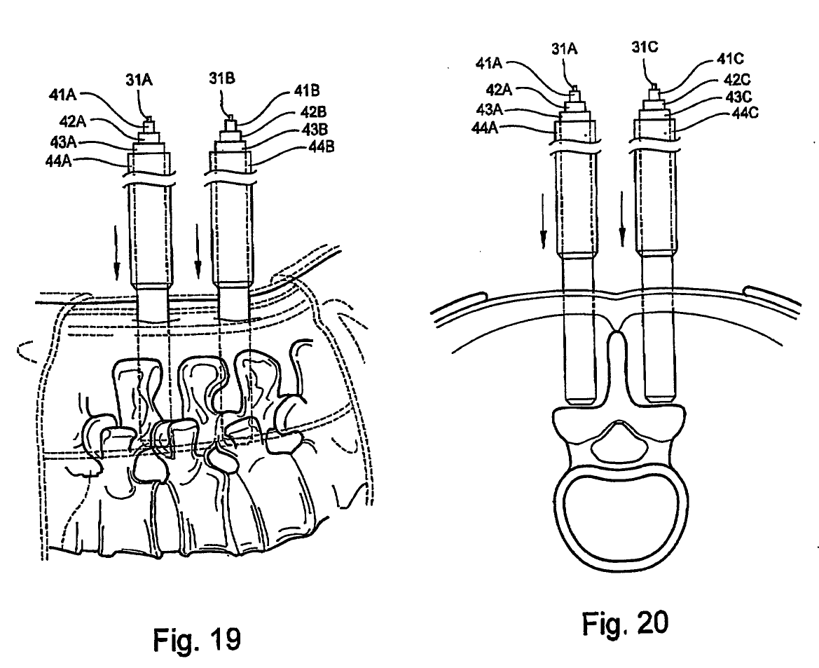

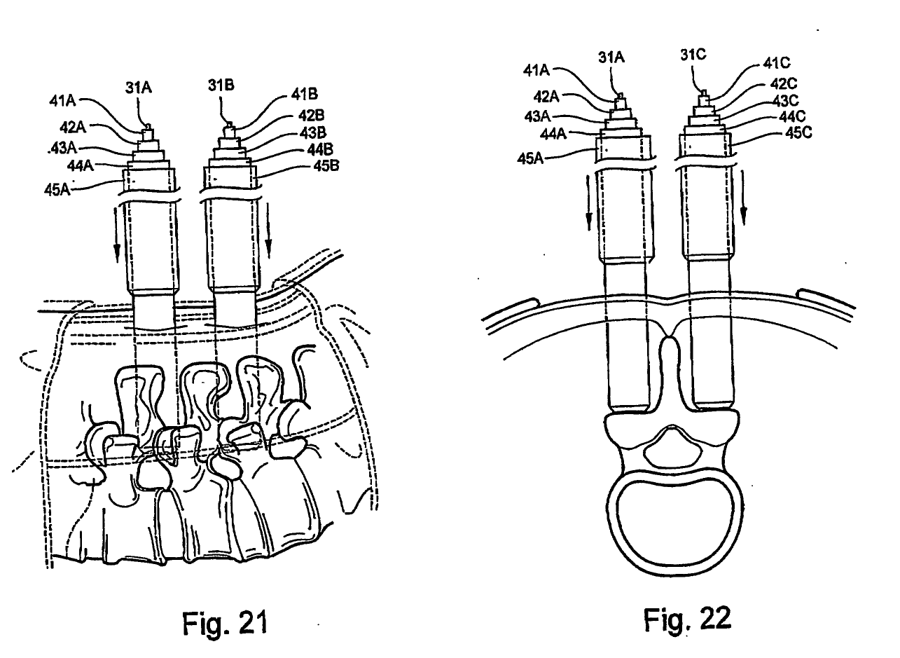

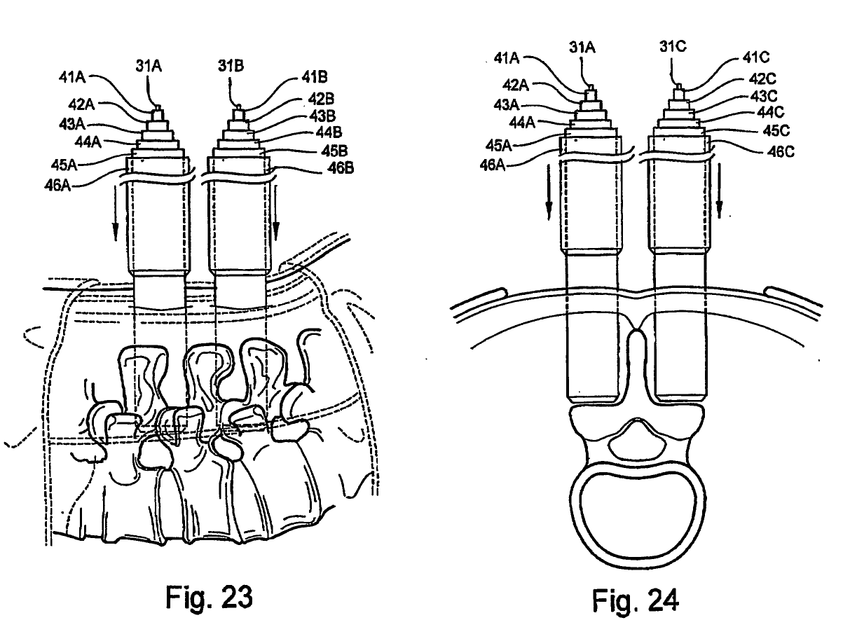

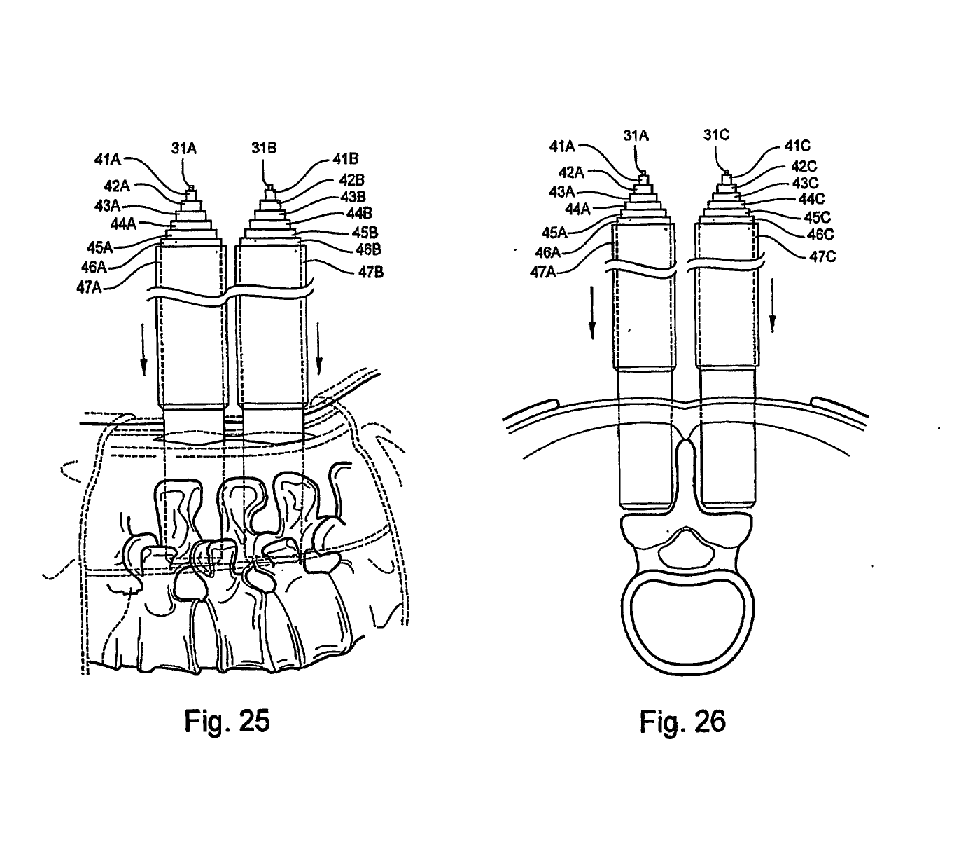

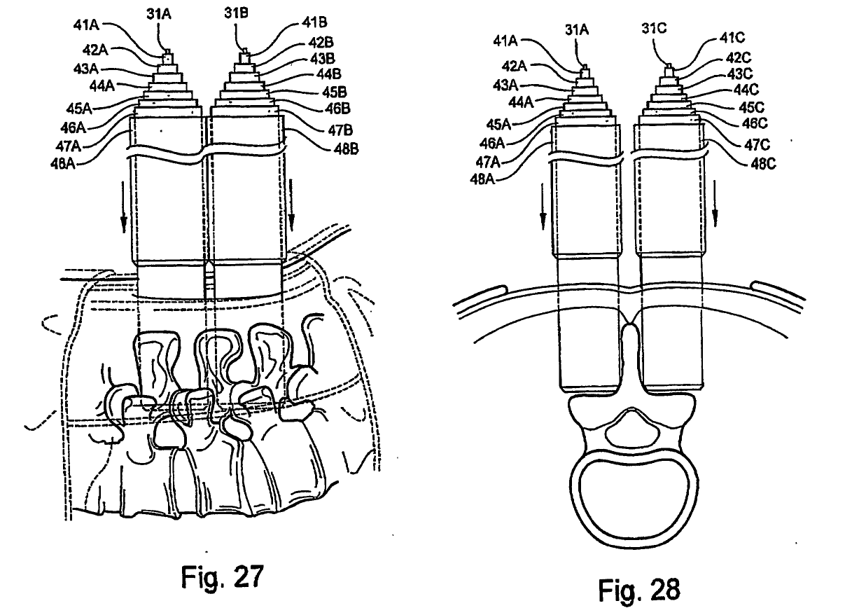

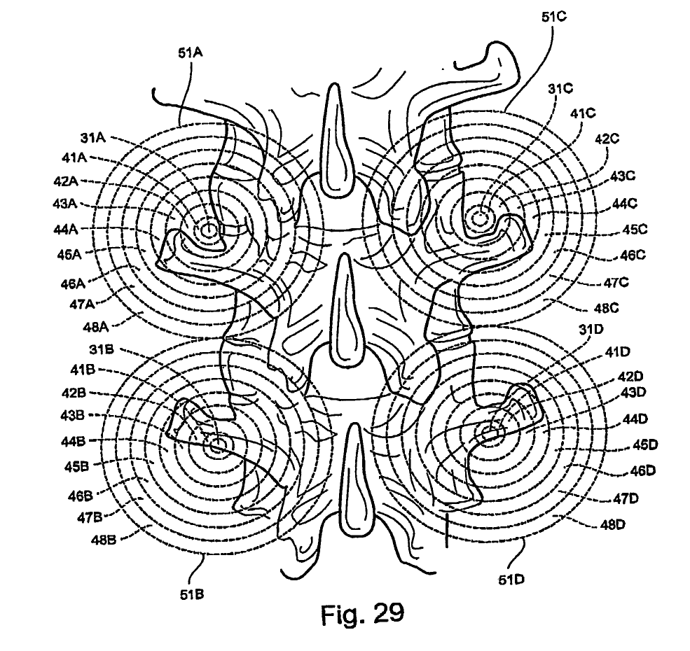

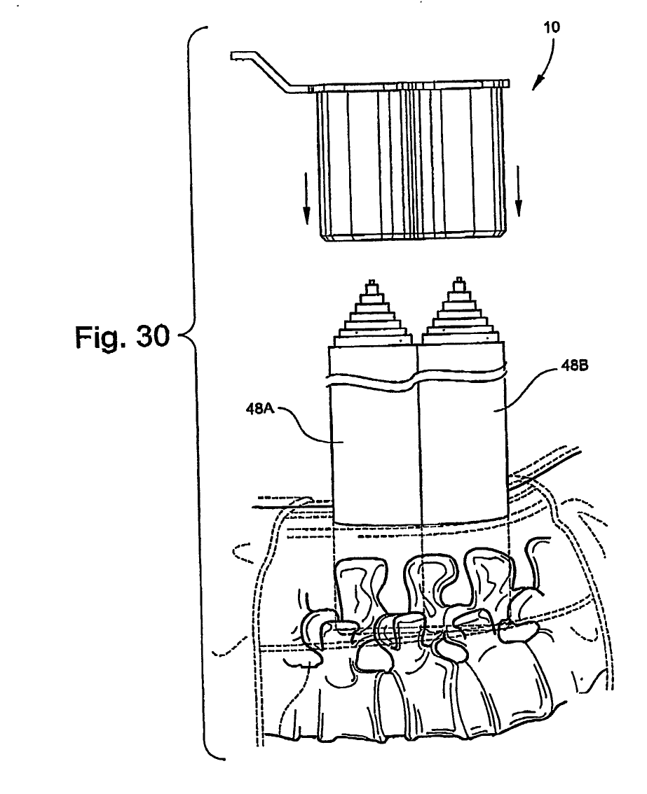

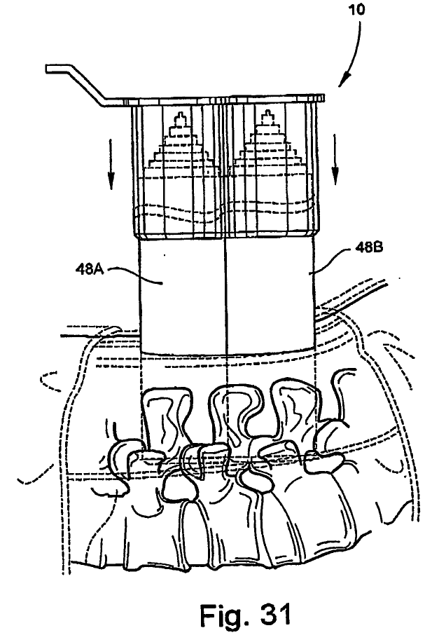

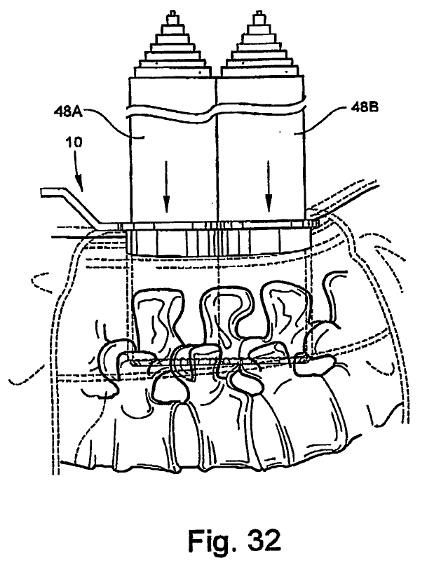

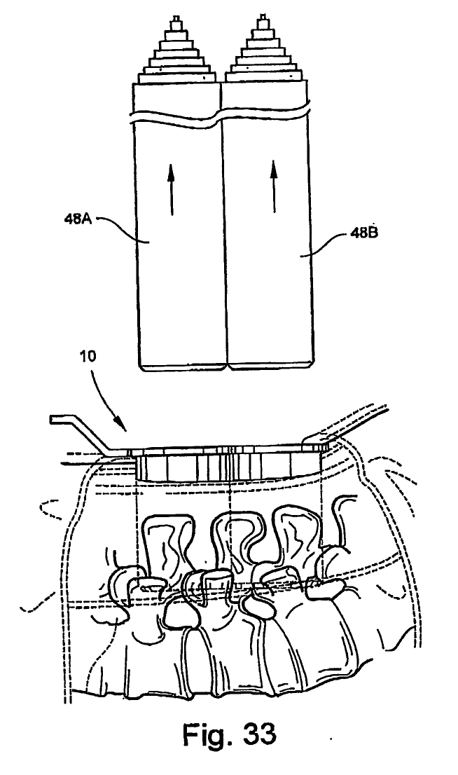

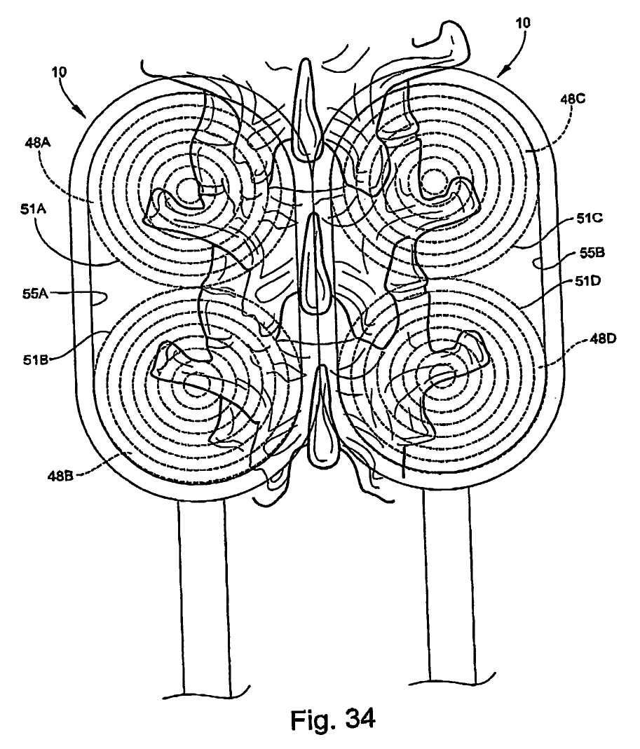

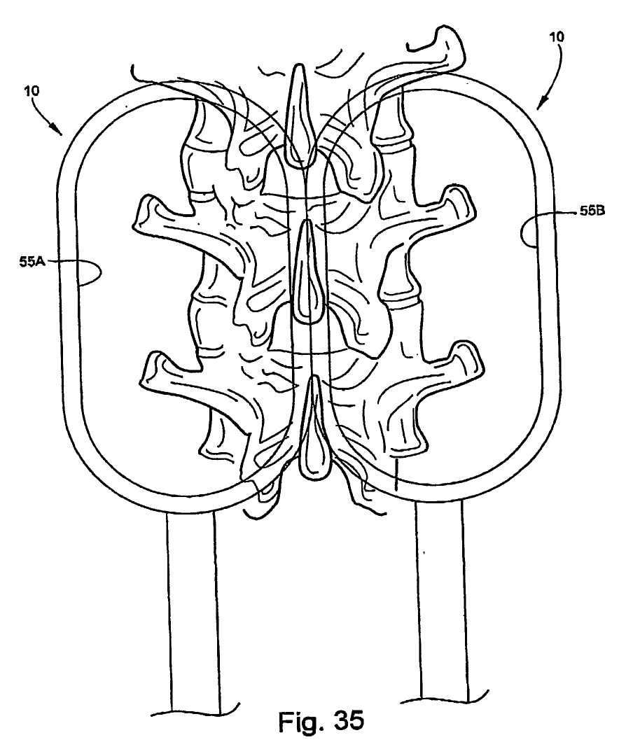

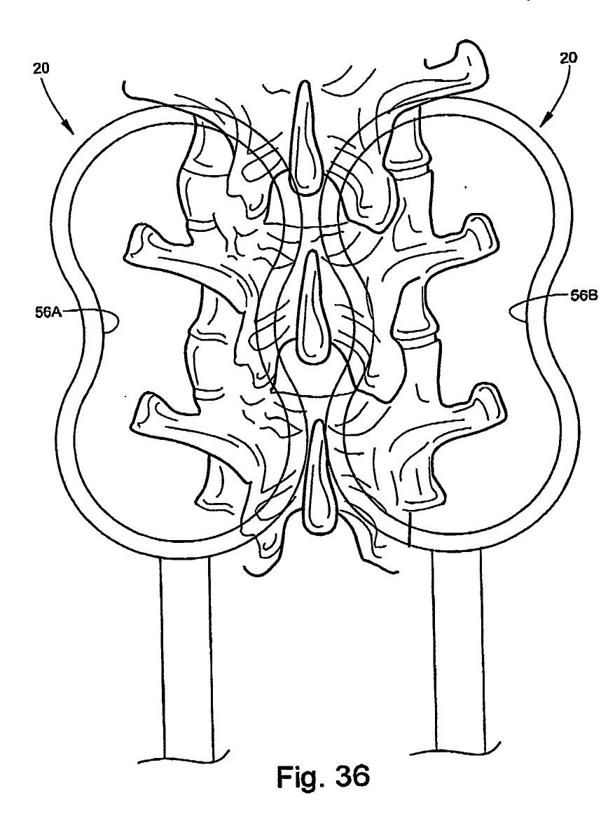

FIGS. 9-33 are sequential views demonstrating installation of the surgical retractor 10 in the patient. Referring to FIGS. 9-12, after prepping the patient, surgery begins by inserting pairs of thin guide wires 31A, 31B and 31C, 31D (31D not shown) through the skin at respective insertion sites on both sides of the spine “S” until they touch the bone at the operating sites. As shown in FIGS. 13-28, an assembly of progressively larger dilators 41A-48A & 41B-48B and 41C-48C & 41D-48D (41D-48D not shown) are then brought down on top of one another following each of the guide wires 31, 32. The dilators 41A-48A, 41B-48B, 41C-48C, and 41D-48D serve to separate and stretch the muscle at the operating sites with minimal cutting. Upon placement of the eighth dilator 48A, 48B, 48C, 48D in each assembly, the muscles are stretched to form pairs of adjacent openings 51A, 51B and 51C, 51D (See FIG. 29) of approximately 25 mm in diameter each. The outside diameter of the eighth dilator 48A, 48B, 48C, 48D in each assembly is preferably 25 mm. The present retractors 10 are then placed over the eighth dilators 48A, 48B and 48C, 48D, respectively, as shown in FIGS. 30-32, in order to hold back the muscles as the guide wires 31A, 31B, 31C, 31D and all of the dilators 41A-48A, 41B-48B, 41C-48C, 41D-48D are subsequently removed from the body, as shown in FIG. 33. This procedure is performed on both sides of the spine “S”. The surgical retractors 10 form enlarged operating portals 55A, 55B to the spine “S”, as shown in FIGS. 34 and 35, and are preferably held in place by respective mechanical arms (not shown) attached to the operating table. FIG. 36 illustrates the operating portals 56A and 56B formed using multiple side by side serial dilators, as previously described, and incorporating the second embodiment of the present retractor 20.

Referring again to FIGS. 2 and 3, according to one embodiment, the width dimension (W) of the surgical retractor 10 is approximately 50 mm and the depth dimension (D) approximately 25 mm—both measurements being taken from an inside surface of the sleeve 11. The height (H) of the surgical retractor 10 will vary depending on the size of the patient, but is generally between 40-80 mm. The sleeve wall thickness is nominal—preferably, less than 1 mm. Each portal 55A, 55B is preferably formed in less than 3 minutes. For the surgical retractor 20, the width dimension is likewise approximately 50 mm and the maximum depth dimension approximately 25 mm. The height of the surgical retractor 20 is also between 40-80, and the wall thickness less than 1 mm.

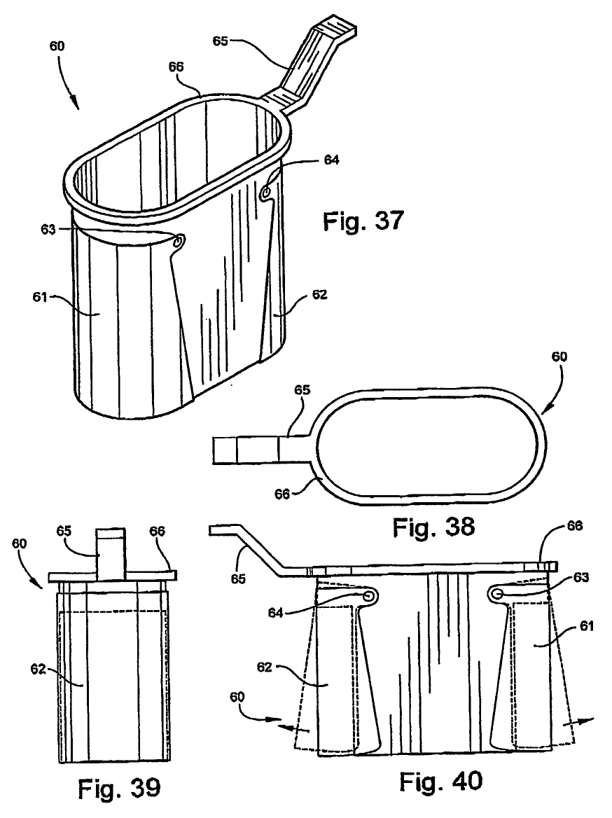

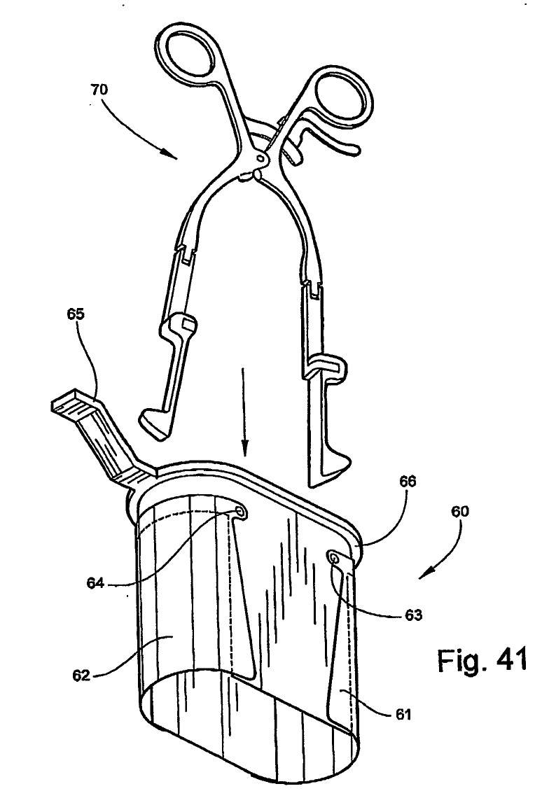

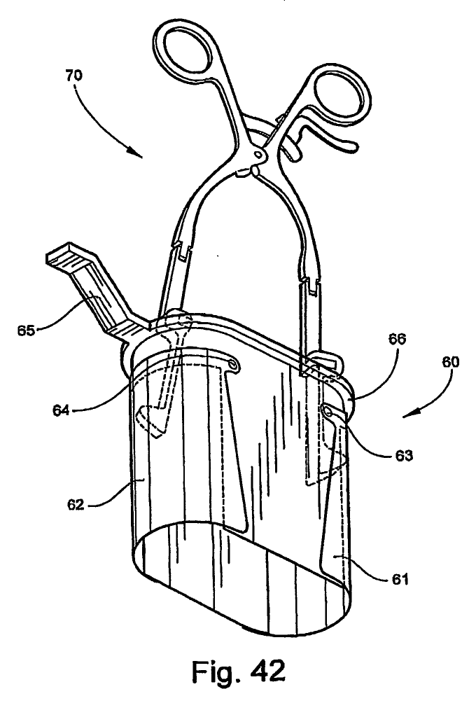

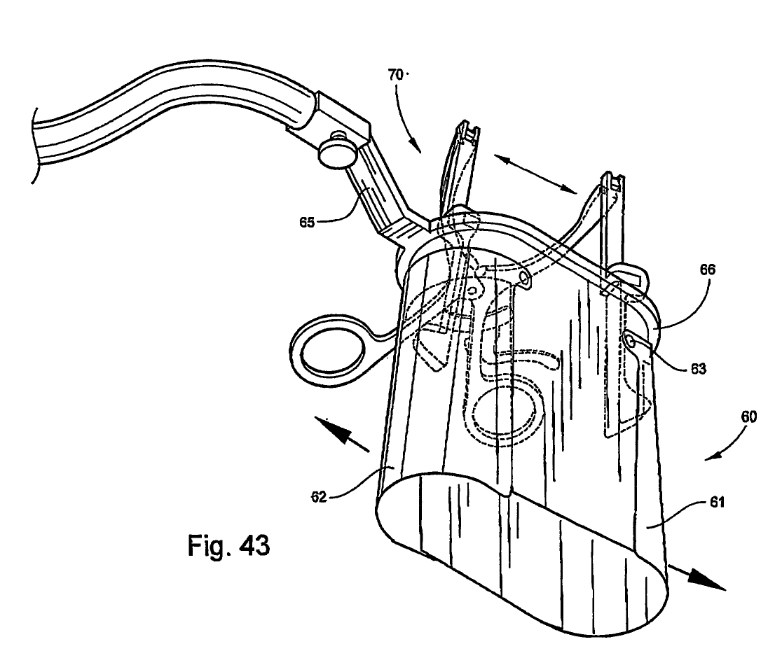

FIGS. 37-43 illustrate a third embodiment of the present retractor 60. According to this embodiment, the curved end walls 61 and 62 of the retractor 60 are hinged at respective points 63 and 64 to allow expansion of the operating portal inside the patient. Any suitable medical instrument 70, shown in FIGS. 41-43, may be used to engage and pivot the end walls 61 and 62 outwardly at respective hinge points 63, 64. Like the retractors 10, 20 described above, the surgical retractor 60 preferably includes a handle 65 and reinforcing flange 66 formed around its top perimeter to help prevent collapsing during use.

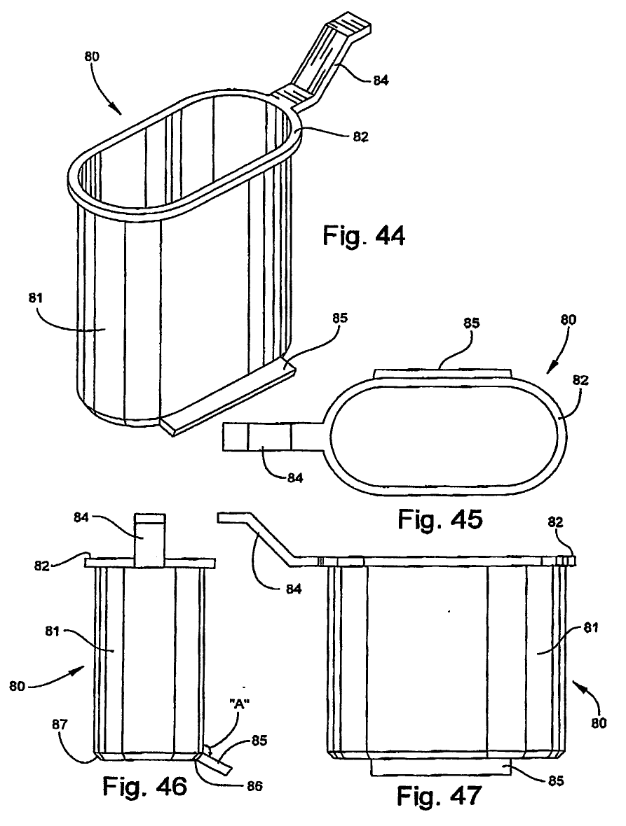

A fourth embodiment of the present surgical retractor 80 is illustrated in FIGS. 44-47. Like the surgical retractor 10, the retractor 80 comprises an open-ended, rigid, generally oval-shaped hollow sleeve 81. A reinforced perimeter flange 82 extends around an open top of the sleeve 80, and provides a clamping area for securing the surgical retractor 80 in a fixed position once placed inside the patient at the operating site. The surgical retractor 80 also includes a convenient handle 84 to facilitate its insertion and manipulation by the surgeon at the operating site, and prior to clamping.

To help clear the operating portal formed by the surgical retractor 80 after insertion in the patient, an outward-projecting muscle wall 85 is formed adjacent an open bottom end of the sleeve 81. Preferably, the muscle wall 85 extends at an angle “A” of between 30 and 120 degrees to the side wall 86 of the sleeve 81, as indicated in FIG. 46. The muscle wall 85 projects from the side wall 86 a distance of 5-10 mm. The opposite side wall 87 of the sleeve 81 is preferably beveled at its bottom end, and has no muscle wall or other projection which would otherwise interfere with the bone at the operating site. When properly placed inside the patient, the muscle wall 85 of the surgical retractor 80 is located away from the spine while the side wall 87 of the sleeve 81 is located proximate the spine. The preferred dimensions of the surgical retractor 80 are identical to those mentioned above for the retractor 10.

Embodiments of an improved surgical retractor are described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

I claim:

1. A surgical retractor, comprising:
an open-ended hollow sleeve adapted for inserting into a body of a patient at an operating site to hold back surrounding muscle and body tissue, said sleeve defining an enlarged operating portal sufficient to allow entry and manipulation of surgical instruments inside the body; and
said sleeve having a width dimension of at least 50 mm, the width dimension accommodating multiple side by side dilators adapted for stretching the muscle and body tissue at the operating site, such that said surgical retractor is inserted into the body over the multiple dilators and the dilators being subsequently removed to establish the enlarged operating portal.

2. A surgical retractor according to claim 1, wherein said sleeve has a generally oval cross-section.

3. A surgical retractor according to claim 1, wherein said sleeve has a generally hourglass-shaped cross-section.

4. A surgical retractor according to claim 1, wherein the width dimension of said sleeve is at least two times a depth dimension of said sleeve.

5. A surgical retractor according to claim 1, wherein said sleeve has a maximum depth dimension of at least 25 mm.

6. A surgical retractor according to claim 1, wherein said sleeve has a length of between 40 and 80 mm.

7. A surgical retractor according to claim 1, and comprising a handle formed with said sleeve for manipulating said surgical retractor inside the body of the patient.

8. A surgical retractor according to claim 1, wherein said sleeve is constructed of a rigid shape-retaining material.

9. A surgical retractor according to claim 1, and comprising an outward-projecting muscle wall located adjacent an open bottom of said sleeve, and adapted to further hold back surrounding muscle and body tissue at the operating site.

10. A surgical retractor, comprising:
an open-ended hollow sleeve adapted for inserting into a body of a patient at an operating site to hold back surrounding muscle and body tissue, said sleeve defining an enlarged operating portal sufficient to allow entry and manipulation of surgical instruments inside the body; and
said sleeve having a width dimension and a depth dimension, the width dimension being at least two times the depth dimension to accommodate multiple side by side dilators adapted for stretching the muscle and body tissue at the operating site, such that said surgical retractor is inserted into the body over the multiple dilators, and the dilators being subsequently removed to establish the enlarged operating portal.

11. A surgical retractor according to claim 10, wherein said sleeve has a generally oval cross-section.

12. A surgical retractor according to claim 10, wherein said sleeve has a generally hourglass-shaped cross-section.

13. A surgical retractor according to claim 10, wherein said sleeve has a maximum depth dimension of at least 25 mm.

14. A surgical retractor according to claim 10, wherein said sleeve has a length of between 40 and 80 mm.

15. A surgical retractor according to claim 10, and comprising a handle formed with said sleeve for manipulating said surgical retractor inside the body of the patient.

16. A method of forming an enlarged operating portal in a body of a patient, said method comprising the steps of:
inserting a first dilator into the body at an insertion site;
inserting a second dilator into the body at an adjacent insertion site;
bringing down an open-ended hollow sleeve over the first and second dilators, and into the body at the insertion sites;
removing the first and second dilators from the body, such that the hollow sleeve defines an enlarged and stable operating portal sufficient to allow entry and manipulation of surgical instruments inside the body at an operating site.

17. A method according to claim 16, and comprising inserting a guide wire through the skin for guiding insertion of the first dilator into the body.

18. A method according to claim 16, and comprising inserting a guide wire through the skin for guiding insertion of the second dilator into the body.

19. A method of forming an enlarged operating portal in a body of a patient, said method comprising the steps of:
inserting a first assembly of progressively larger serial dilators into the body at an insertion site;
inserting a second assembly of progressively larger serial dilators into the body at an adjacent insertion site;
bringing down an open-ended hollow sleeve over outermost ones of the first and second assemblies of serial dilators, and into the body at the insertion sites;
removing the first and second assemblies of serial dilators from the body, such that the hollow sleeve defines an enlarged and stable operating portal sufficient to allow entry and manipulation of surgical instruments inside the body at an operating site.

20. A method according to claim 19, and comprising inserting first and second guide wires through the skin for guiding insertion of respective first and second serial dilators into the body.
